(19) United States
(12) Patent Application Publication
(10) Pub. No.: US 2022/0274094 A1
(43) Pub. Date: Sep. 1, 2022

(54) SELECTIVE PRODUCTION OF ETHYLENE FROM METHANE

(71) Applicant: SABIC Global Technologies B.V., Bergen op Zoom (NL)

(72) Inventor: Muhammad H. HAIDER, Riyadh (SA)

(21) Appl. No.: 17/626,997

(22) PCT Filed: Jul. 9, 2020
(86) PCT No.: PCT/IB2020/056469

§ 371 Date: Jan. 13, 2022

Related U.S. Application Data
(60) Provisional application No. 62/875,469, filed on Jul. 17, 2019.

(51) Int. Cl. B01J 23/882; B01J 23/30; B01J 37/03; B01J 37/18; C07C 1/24; C07C 29/151; C07C 29/156; C07C 29/80

(52) U.S. Cl. CPC B01J 23/882; B01J 23/30; B01J 37/03; B01J 37/18; C07C 1/24; C07C 29/1518; C07C 29/156; C07C 29/80; C07C 2521/04; C07C 2523/04; C07C 2523/30

(57) ABSTRACT

Disclosed are processes for producing ethylene. The processes can include contacting a first stream containing methane with an oxidant and oxidizing at least a portion of the methane under conditions suitable to produce a second stream containing carbon monoxide (CO) and hydrogen (H₂), contacting the second stream with a CO hydrogenation catalyst under conditions suitable to produce a third stream containing methanol and ethanol, obtaining a fourth stream containing the ethanol and a fifth stream containing the methanol from the third stream, and contacting the fourth stream with an ethanol dehydration catalyst under conditions suitable to dehydrate at least a portion of the ethanol and produce a products stream containing ethylene.

### CROSS REFERENCE TO RELATED APPLICATIONS

This application claims the benefit of priority of U.S. Provisional Patent Application No. 62/875,469, filed Jul. 17, 2019, which is hereby incorporated by reference in its entirety.

### FIELD OF INVENTION

The invention generally concerns compositions, processes, and systems for production of ethylene. In particular, the invention concerns compositions, processes, and systems for the selective production of ethylene from methane via converting methane into syngas, producing ethanol from the syngas via carbon monoxide (CO) hydrogenation, and converting the ethanol into ethylene via ethanol dehydration.

### BACKGROUND OF THE INVENTION

Ethylene is an important raw material for multiple end products like polymers, rubbers, plastics etc. Currently there exists a big gap in ethylene demand and production and it is expected that demand for ethylene will continue to grow.

There are many types of processes known to produce ethylene. In one example, WO 2016/069389 discloses a process for converting a mixture of methane and ethane to syngas and ethylene. The process includes two distinct reactions—methane to syngas and ethane to ethylene—occurring concurrently and promoted by a single catalyst. Another example includes ethylene production from CO hydrogenation. However, this process remains inefficient either due to insufficient processing conditions, a lack of suitable catalysts, and/or convoluted schematics for implementing such processes (see, e.g., Liu et al. BIORESOURCE TECHNOLOGY, 2014 (151), pp 69-77; Gupta et al. ACS Catal, 2011, 1 (6), pp 641-656; Lee et al., Applied Catal A, 2014 (480), pp 128-133; Surisetty et al., Applied Catal A. 2009 (365), pp 243-251).

### SUMMARY OF THE INVENTION

A discovery has been made that provides a solution to at least some of the aforementioned problems associated with the production of ethylene. A solution of the present invention can include the conversion of methane to ethylene through a sequence of steps that can result in high selectivity towards ethylene production in a reasonably cost-efficient manner. The sequence of steps can include (a) conversion of methane into carbon monoxide (CO) and hydrogen (H₂) (e.g., syngas); (b) conversion of CO into methanol and ethanol via CO hydrogenation; (c) conversion of ethanol into ethylene via ethanol dehydration. In one particular instance, it was discovered that a crystalline cobalt molybdenum catalyst can be used as a CO hydrogenation catalyst, which can produce ethanol with a high selectivity, which can further enhance the efficiency of the overall methane to ethylene conversion process.

In one aspect of the present invention, a process for producing ethylene is described. The process can include steps (a)-(d). In step (a) a first stream containing methane can be contacted with an oxidant and at least a portion of the methane can be oxidized under conditions suitable to produce a second stream containing carbon monoxide (CO) and hydrogen (H₂). In step (b) the second stream can be contacted with a CO hydrogenation catalyst under conditions suitable to produce a third stream comprising methanol and ethanol, by hydrogenating the CO with the H₂. In step (c) a fourth stream containing the ethanol and a fifth stream containing the methanol can be obtained from the third stream. The third stream can be separated by one or more separation steps to obtain the fourth stream and the fifth stream. In step (d) the fourth stream can be contacted with an ethanol dehydration catalyst under conditions suitable to dehydrate at least a portion of the ethanol and produce a products stream containing ethylene. In some aspects, the third stream can further contain C2-C7 paraffins and carbon dioxide (CO₂) and the process can further include steps (i) and (ii). In step (i) the third stream can be separated to obtain a first intermediate stream containing the methanol and ethanol and a second intermediate stream containing the C2-C7 paraffins and carbon dioxide (CO₂). In step (ii) the first intermediate stream can be separated to obtain the fourth stream and the fifth stream. In some aspects, the CO hydrogenation catalyst can include a crystalline cobalt molybdenum catalyst. In some aspects, the crystalline cobalt molybdenum catalyst can include a monoclinic cobalt molybdenum catalyst. A monoclinic cobalt molybdenum catalyst can have a monoclinic crystalline system (monoclinic crystalline system or structure can be used interchangeably in this specification). In some aspects, the monoclinic cobalt molybdenum catalyst can be a monoclinic cobalt molybdenum oxide. In some aspects, the monoclinic cobalt molybdenum oxide can be CoₓMo_{y}O_{z}, with x ranging from 0.5 to 1.5, preferably 0.9 to 1.1, y ranging from 0.5 to 1.5, preferably 0.9 to 1.1, and z can be a value that balances the valencies of Co and Mo. In certain aspects z can be 3.5 to 4.5, preferably 3.9 to 4.1. In some particular aspect, the monoclinic cobalt molybdenum oxide can contain α-CoMoO4 and β-CoMoO4 and the wt. % ratio of α-CoMoO4 to β-CoMoO4 can be 15:85 to 35:65, preferably 20:80 to 30:70. In some aspects, the CO hydrogenation catalyst can be activated, prior to contacting the catalyst with the second stream. The catalyst can be activated by reduction with hydrogen (H₂). In certain instances, the activation process can include reducing the catalyst with a stream containing hydrogen (H₂) at a temperature 200° C. to 500° C., at a GHSV of 1000 h⁻¹ to 3000 h⁻¹, and/or at pressure 25 bar to 90 bar for 8 h to 20 h. The oxidant in step (a) can be steam, oxygen (O₂), CO₂ or any combination thereof. The oxidation of the methane in step (a) can be catalyzed using a methane oxidation catalyst. In some aspects, the methane oxidation catalyst can include one or more metals on a support. The one or more metals can be one or more of La, Ni, Ru, Rh, Pd, Ir or Pt. The support can be alumina, silica, zirconia, ceria, titania, magnesium oxide, magnesium aluminate or any combination thereof. In some aspects, the methane oxidation catalyst can contain a promoter. In some aspects the promoter can be an alkali metal, and/or an alkaline earth metal. In some aspects the promoter can be Li, Na, K, or a combination thereof. The methane oxidation conditions in step (a) can include a pressure of 0 to 180 bar, GHSV of 5000 to 15000 and/or a temperature of 500 to 1600° C. In some aspects, the methane in the first stream can be obtained from a refinery, petroleum by product, or renewable feedstock or combinations thereof. The molar ratio of the H₂ and CO in the second stream can be 0.5:1 to 3:1, preferably 0.8:1 to 1.2:1. The contacting conditions in step (b) can include a pressure of 25 to 90 bar, GHSV of 1000 to 3000 h⁻¹ and/or a temperature of 150 to 450° C. Combined mol. % of the methanol and ethanol in the third stream can be at least 50 mol. %. In some aspects, the combined mol. % of methanol and ethanol in the third stream can be 50 mol. % to 75 mol. %. In some aspects, third stream can contain 20 mol. % to 40 mol. %, preferably 25 mol. % to 40 mol. %, more preferably 30 mol. % to 35 mol. % methanol; 20 mol. % to 40 mol. %, preferably 25 mol. % to 40 mol. wt. %, more preferably 30 mol. % to 35 mol. % ethanol; 5 mol. % to 25 mol. % C2 to C7 paraffins and 10 mol. % to 20 mol. % CO₂. Combined selectivity of propanol and butanol obtained from CO hydrogenation in step (b) can be less than 20%, preferably less than 15%, more preferably less than 10%. In some aspects, selectivity of propanol obtained in step (b) can be less than 10%, preferably less than 7%, more preferably less than 5%. In some aspects, selectivity of butanol obtained in step (b) can be less than 10%, preferably less than 7%, more preferably less than 5%. In some aspects, in step (i) the third stream can be separated by traditional gas liquid separation. In some aspects, in step (i) the third stream can be separated by distillation using a distillation column and the first intermediate stream can be obtained as a bottom distillate product and the second intermediate stream can be obtained as a top distillate product. In some aspects, in step (ii) the first intermediate stream can be separated by distillation using a distillation column and the fourth stream can be obtained as a bottom distillate product and the fifth stream can be obtained as a top distillate product. The ethanol dehydration conditions in step (d) can include a pressure of 0 to 90 bar, GHSV of 1000 to 3000 h⁻¹ and/or a temperature of 105 to 450° C. The ethanol dehydration catalyst in step (d) can be an acid type catalyst. In some aspects the acid type catalyst can be cesium doped silicotungstic acid supported on alumina. The products stream obtained in step (d) can contain 90 wt. % to 100 wt. %, preferably 95 wt. % to 100 wt. %, more preferably 98 wt. % to 100 wt. % ethylene.

Other embodiments of the invention are discussed throughout this application.

Any embodiment discussed with respect to one aspect of the invention applies to other aspects of the invention as well and vice versa. Each embodiment described herein is understood to be embodiments of the invention that are applicable to other aspects of the invention. It is contemplated that any embodiment discussed herein can be implemented with respect to any method or composition of the invention, and vice versa. Furthermore, compositions and systems of the invention can be used to achieve methods of the invention.

The following includes definitions of various terms and phrases used throughout this specification.

The term “monoclinic crystal structure” refers to a crystal that is described by three unequal-length vectors that form a rectangular prism with a parallelogram base, wherein two of said vectors are substantially perpendicular, while the third vector meets the other two at an angle other than 90°.

C2-C7 paraffins refers to paraffin hydrocarbons having a carbon number 2 to 7 (e.g. ethane, propane, butane, pentane, hexane, heptane etc.).

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %,” “vol. %,” or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%. “Essentially free” is defined as having no more than about 0.1% of a component.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Selectivity of a compound in a reaction is defined as: Selectivity of a compound one=(moles of compound one produced/total moles produced)*100

The process and systems of the present invention can “comprise,” “consist essentially of,” or “consist of” particular ingredients, components, compositions, steps, etc. disclosed throughout the specification. With respect to the transitional phrase “consisting essentially of,” in one non-limiting aspect, a basic and novel characteristic of the processes and the systems of the present invention are their abilities to produce ethylene from methane using intermediate steps CO and H₂ formation from methane, methanol and ethanol production from CO hydrogenation, and ethanol dehydration to produce ethylene. The CO hydrogenation step can have a relatively high selectivity for methanol and ethanol (e.g., combined selectivity of methanol and ethanol of at least 50%), which can be advantageous for the production of ethylene.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

FIG. 1: Schematic of an example of the present invention to produce ethylene.

FIG. 2: Thermal Gravimetric Analysis of crystalline cobalt molybdenum catalyst.

FIG. 3: X-ray power diffraction of crystalline cobalt molybdenum catalyst.

FIG. 4: Raman spectrum of crystalline cobalt molybdenum catalyst.

FIG. 5: CO conversion percentage obtained with cobalt molybdenum catalyst.

### DETAILED DESCRIPTION OF THE INVENTION

A discovery has been made that provides solutions to at least some of the problems associated with the production of ethylene from a methane containing C1 hydrocarbon feedstock. The solution is premised on producing CO and H2 from the C1 hydrocarbon feedstock, hydrogenating the CO with the H2 (or supplemental) using a CO hydrogenation catalyst to produce ethanol with high selectivity, optionally separating the ethanol from the other products produced during CO hydrogenation, and dehydrating the ethanol to produce ethylene.

These and other non-limiting aspects of the present invention are discussed in further detail in the following paragraphs with reference to the figures.

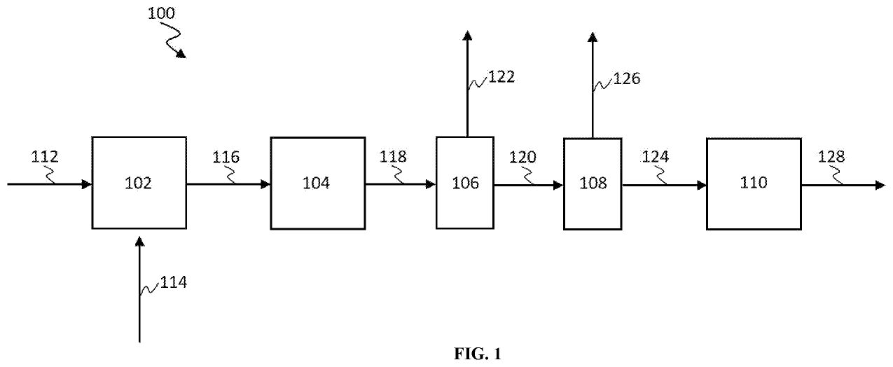

Referring to FIG. 1, one example of a system and process of the present invention for producing ethylene is described. System 100 can include a methane oxidizing unit 102, a CO hydrogenation unit 104, a first separation unit 106, a second separation unit 108, and an ethanol dehydration unit 110.

A first stream 112 containing methane can be fed to the methane oxidizing unit 102. In the methane oxidizing unit 102 the methane can get oxidized by an oxidant to produce CO and H₂. The oxidant can be steam, O₂, CO₂ or any combination thereof. The oxidant can be fed to the methane oxidizing unit 102 as a separate feed 114 or it can be mixed with the first stream 112 and fed to the methane oxidizing unit 102 as a single feed (not shown). The methane oxidation conditions in the methane oxidizing unit 102 can include a pressure of 0 bar to 180 bar, or at least any one of, equal to any one of, or between any two of 0 bar, 15 bar, 30 bar, 45 bar, 60 bar, 75 bar, 90 bar, 105 bar, 120 bar, 135 bar, 150 bar, 165 bar and 180 bar, GHSV of 5000 h⁻¹ to 15000 h⁻¹ or at least any one of, equal to any one of, or between any two of 5000 h⁻¹, 6000 h⁻¹, 7000 h⁻¹, 8000 h⁻¹, 9000 h⁻¹, 10000 h⁻¹, 1000 h⁻¹, 12000 h⁻¹, 13000 h⁻¹, 14000 h⁻¹ and 15000 h⁻¹ and/or a temperature of 500° C. to 1600° C. or at least any one of, equal to any one of, or between any two of 500° C., 600° C., 700° C., 800° C., 900° C., 1000° C., 1100° C., 1200° C., 1300° C., 1400° C., 1500° C. and 1600° C. In some aspects, the methane oxidizing unit 102 can contain a methane oxidation catalyst (not shown) and the methane oxidation can be catalyzed by the methane oxidation catalyst. In some aspects, the methane oxidizing unit 102 can be a part of a chemical looping system (not shown), and the methane can be oxidized via chemical looping, wherein the oxidant can be provided to the methane by an oxidized methane oxidation catalyst and/or oxygen transfer agent. The methane oxidation catalyst can contain one or more metals on a support. The one or more metals can be one or more of La, Ni, Ru, Rh, Pd, Ir or Pt. The support can be alumina, silica, zirconia, ceria, titania, magnesium oxide, magnesium aluminate or a combination thereof. In some aspects, the methane oxidation catalyst can contain a promoter. In some aspects the promoter can be an alkali metal, and/or an alkaline earth metal. In some aspects the promoter can be Li, Na, K, or a combination thereof Non-limiting examples of methane oxidation catalysts that can be used in the context of the present invention can include LaNiAl₂O₃, LiLaNiAl₂O₃, NaLaNiAl₂O₃, KLaNiAl₂O₃, or a methane oxidation catalyst as described in Khalesi et. al., Ind. Eng. Chem. Res., 2008, 47, 5892-5898.

A second stream 116 containing at least a portion of the CO and H₂ produced from methane oxidation can enter the CO hydrogenation unit 104. The H₂ and CO molar ratio in the second stream can be 0.5:1 to 3:1 or at least any one of, equal to any one of, or between any two of 0.5:1, 0.8:1, 1:1, 1.2:1, 1.5:1, 2:1, 2.5:1, and 3:1. In the CO hydrogenation unit 104 the second stream 116 can be contacted with a CO hydrogenation catalyst (not shown) to hydrogenate the CO with the H₂ and produce methanol, ethanol, C2-C7 paraffins and CO₂. The combined selectivity of the methanol and ethanol can be 50% to 75% or at least any one of, equal to any one of, or between any two of 50 %, 51 %, 52 %, 53 %, 54 %, 55 %, 56 %, 57 %, 58%, 59 %, 60 %, 61%, 62 %, 63%, 64 %, 65 %, 66 %, 67 %, 68 %, 69 %, 70 %, 71%, 72%, 73%, 74%, and 75%. In some particular aspects, the selectivity of the methanol can be 20% to 40% or at least any one of, equal to any one of, or between any two of 20%, 21%, 22%, 23%, 24%, 25%, 26%, 27%, 28%, 29%, 30%, 31%, 32%, 33%, 34%, 35%, 36%, 37%, 38%, 39% and 40%. In some particular aspects, the selectivity of the ethanol can be 20% to 40% or at least any one of, equal to any one of, or between any two of 20%, 21%, 22%, 23%, 24%, 25%, 26%, 27%, 28%, 29%, 30%, 31%, 32%, 33%, 34%, 35%, 36%, 37%, 38%, 39% and 40%. In some particular aspects, the selectivity of the C2 to C7 paraffins can be 5% to 25% or at least any one of, equal to any one of, or between any two of 5%, 6%, 7%, 8%, 9%, 10%, 11%, 12%, 13%, 14%, 15%, 16%, 17%, 18%, 19%, 20%, 21%, 22%, 23%, 24%, and 25%. In some particular aspects, the selectivity of the CO₂ can be 10% to 20% or at least any one of, equal to any one of, or between any two of 10%, 11%, 12%, 13%, 14%, 15%, 16%, 17%, 18%, 19%, and 20%. In some aspects, the CO conversion can be 20% to 40% or at least any one of, equal to any one of, or between any two of 20%, 21%, 22%, 23%, 24%, 25%, 26%, 27%, 28%, 29%, 30%, 31%, 32%, 33%, 34%, 35%, 36%, 37%, 38%, 39% and 40%. The CO hydrogenation conditions can include a pressure 25 bar to 90 bar or at least any one of, equal to any one of, or between any two of 25 bar, 35 bar, 45 bar, 55 bar, 65 bar, 75 bar, 85 bar, and 90 bar, GHSV 1000 h⁻¹ to 3000 h⁻¹ or at least any one of, equal to any one of, or between any two of 1000 h¹, 1100 h⁻¹, 1200 h⁻¹, 1300 h⁻¹, 1400 h⁻¹, 1500 h⁻¹, 1600 h⁻¹, 1700 h⁻¹, 1800 h⁻¹, 1900 h⁻¹, 2000 h⁻¹, 2100 h⁻¹, 2200 h⁻¹, 2300 h⁻¹, 2400 h⁻¹, 2500 h⁻¹, 2600 h⁻¹, 2700 h⁻¹, 2800 h⁻¹, 2900 h⁻¹, and 3000 h⁻¹ and/or a temperature 150° C. to 450° C. or at least any one of, equal to any one of, or between any two of 150° C., 200° C., 250° C., 300° C., 350° C., 400° C., and 450° C. In some aspects, the CO hydrogenation catalyst can be activated, prior to contacting the catalyst with the second stream 116. In some aspects, the CO hydrogenation catalyst can be contacted with a stream containing H₂ at a temperature 200° C. to 500° C. or at least any one of, equal to any one of, or between any two of 200° C., 250° C., 300° C., 350° C., 400° C., 450° C. and 500° C., at a GHSV 1000 h⁻¹ to 3000 h⁻¹ or at least any one of, equal to any one of, or between any two of 1000 h⁻¹, 1100 h⁻¹, 1200 h⁻¹, 1300 h⁻¹, 1400 h⁻¹, 1500 h⁻¹, 1600 h⁻¹, 1700 h⁻¹, 1800 h⁻¹, 1900 h⁻¹, 2000 h⁻¹, 2100 h⁻¹, 2200 h⁻, 2300 h⁻, 2400 h⁻¹, 2500 h⁻¹, 2600 h⁻¹, 2700 h⁻¹, 2800 h⁻¹, 2900 h⁻¹, and 3000 h⁻¹, and/or at a pressure 25 bar to 90 bar or at least any one of, equal to any one of, or between any two of 25 bar, 35 bar, 45 bar, 55 bar, 65 bar, 75 bar, 85 bar, and 90 bar for 8 h to 20 h or at least any one of, equal to any one of, or between any two of 8 h, 10 h 12 h, 14 h, 16 h, 18 h and 20 h to reduce and activate the catalyst. In some aspects, the system 100 can include an off-line secondary CO hydrogenation reactor (not shown) in addition to the on-line primary CO hydrogenation reactor 104. The CO hydrogenation catalyst can be activated and/or regenerated in the secondary CO hydrogenation reactor. Activation and/or regeneration of the CO hydrogenation catalyst in the secondary CO hydrogenation reactor can be performed in parallel to the CO hydrogenation in the primary CO hydrogenation reactor 104. Once regeneration/activation of the CO hydrogenation catalyst in the primary CO hydrogenation reactor becomes necessary, the primary CO hydrogenation reactor can be taken offline and the secondary CO hydrogenation reactor with the activated catalyst can be brought on-line and thereby primary become secondary and the secondary becomes primary CO hydrogenation reactor. The parallel activation process can be repeated to ensure continuous operation of the ethylene production process.

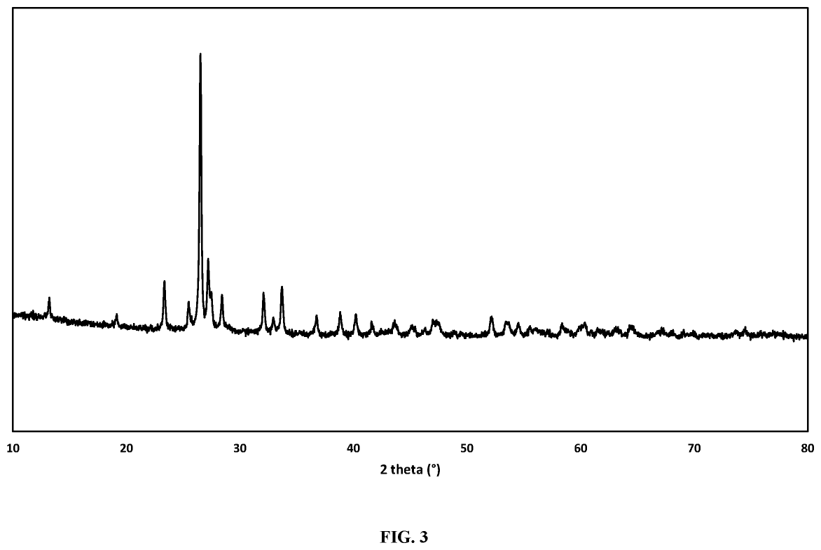

The CO hydrogenation catalyst can be a crystalline cobalt molybdenum catalyst. The crystalline cobalt molybdenum catalyst can include a monoclinic crystalline structure. In some aspects, the monoclinic cobalt molybdenum catalyst can be a monoclinic cobalt molybdenum oxide. In some aspects, the monoclinic cobalt molybdenum oxide can be CoₓMo_{y}O_{z}, where x can be 0.5 to 1.5 or at least any one of, equal to any one of, or between any two of 0.5, 0.6, 0.7, 0.8, 0.9. 1, 1.1, 1.2, 1.3, 1.4 and 1.5, y can be 0.5 to 1.5 or at least any one of, equal to any one of, or between any two of 0.5, 0.6, 0.7, 0.8, 0.9. 1, 1.1, 1.2, 1.3, 1.4 and 1.5, and z can balance the valencies of Co and Mo. In certain aspects, z can be 3.5 to 4.5 or at least any one of, equal to any one of, or between any two of 3.5, 3.6, 3.7, 3.8, 3.9. 4, 4.1, 4.2, 4.3, 4.4 and 4.5. In some particular aspects, the monoclinic cobalt molybdenum oxide can include α-CoMoO₄ and β-CoMoO₄ at a α-CoMoO₄ to β-CoMoO₄ wt. % ratio 15:85 to 35:65 or at least any one of, equal to any one of, or between any two 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66 and 35:65. The catalyst can be a bulk or a supported catalyst, preferably a bulk catalyst. In some aspects the catalyst does not contain a support. In some aspects, the catalyst does not contain a cobalt sulfide, a molybdenum sulfide and/or a metal sulfide. In some aspects, the catalyst does not contain an alkali metal. In some aspects, the catalyst does not contain an alkaline earth metal. In some aspects, the crystalline cobalt molybdenum catalyst can have an X-ray power diffraction pattern as substantially depicted in FIG. 3. In other aspects of the invention, however, other CO hydrogenation catalysts can be used.

A third stream 118 containing at least a portion of the ethanol, methanol, C2-C7 paraffins, and CO₂ obtained from CO hydrogenation can enter the first separation unit 106. In some aspects, the third stream can contain at least any one of, equal to any one of, or between any two of 20 mol. %, 21 mol. %, 22 mol. %, 23 mol. %, 24 mol. %, 25 mol. %, 26 mol. %, 27 mol. %, 28 mol. %, 29 mol. %, 30 mol. %, 31 mol. %, 32 mol. %, 33 mol. %, 34 mol. %, 35 mol. %, 36 mol. %, 37 mol. %, 38 mol. %, 39 mol. % and 40 mol. % methanol; at least any one of, equal to any one of, or between any two of 20 mol. %, 21 mol. %, 22 mol. %, 23 mol.

%, 24 mol. %, 25 mol. %, 26 mol. %, 27 mol. %, 28 mol. %, 29 mol. %, 30 mol. %, 31 mol. %, 32 mol. %, 33 mol. %, 34 mol. %, 35 mol. %, 36 mol. %, 37 mol. %, 38 mol. %, 39 mol. % and 40 mol. % ethanol; at least any one of, equal to any one of, or between any two of 5 mol. %, 6 mol. %, 7 mol. %, 8 mol. %, 9 mol. %, 10 mol. %, 11 mol. %, 12 mol. %, 13 mol. %, 14 mol. %, 15 mol. %, 16 mol. %, 17 mol. %, 18 mol. %, 19 mol. %, 20 mol. %, 21 mol. %, 22 mol. %, 23 mol. %, 24 mol. %, and 25 mol. % C2-C7 paraffins and at least any one of, equal to any one of, or between any two of 10 mol. %, 11 mol. %, 12 mol. %, 13 mol. %, 14 mol. %, 15 mol. %, 16 mol. %, 17 mol. %, 18 mol. %, 19 mol. %, and 20 mol. % CO2.

In the first separation unit 106 the third stream 118 can be separated to obtain a first intermediate stream 120 containing the ethanol and methanol and a second intermediate stream 122 containing the C2-C7 paraffins and CO₂. The separation of the third stream 118 in the first separation unit 106 can be obtained by any suitable methods known in the art e.g., distillation, fractionation, pressure swing adsorption, and the like. In some aspects, the first separation unit 106 can contain a distillation column and the first intermediate stream 120 can be obtained as a bottom distillate product and the second intermediate stream 122 can be obtained as a top distillate product. In some aspects, column operating conditions can include a pressure 0 bar to 5 bar or at least any one of, equal to any one of, or between any two of 0 bar, 1 bar, 2 bar, 3 bar, 4 bar and 5 bar and/or a temperature 25° C. to 35° C. or at least any one of, equal to any one of, or between any two of 25° C., 26° C., 27° C., 28° C., 29° C., 30° C., 31° C., 32° C., 33° C., 34° C. and 35° C.

The first intermediate stream 120 can enter the second separation unit 108. In the second separation unit 108 the first intermediate stream 120 can be separated to obtain a fourth stream 124 containing the ethanol and a fifth stream 126 containing the methanol. The separation of the first intermediate stream 120 in the second separation unit 108 can be obtained by any suitable methods known in the art e.g., distillation, fractionation, pressure swing adsorption, and the like. In some aspects, the second separation unit 108 can contain a distillation column and the fourth stream 124 can be obtained as a bottom distillate product and the fifth stream 126 can be obtained as a top distillate product. In some aspects, column operating conditions can include a pressure 0 bar to 5 bar or at least any one of, equal to any one of, or between any two of 0 bar, 1 bar, 2 bar, 3 bar, 4 bar and 5 bar and/or a temperature 25° C. to 35° C. or at least any one of, equal to any one of, or between any two of 25° C., 26° C., 27° C., 28° C., 29° C., 30° C., 31° C., 32° C., 33° C., 34° C. and 35° C.

The fourth stream 124 can enter the ethanol dehydration unit 110. In the ethanol dehydration unit 110 the fourth stream 124 can be contacted with an ethanol dehydration catalyst (not shown) under conditions suitable to dehydrate at least a portion of the ethanol and produce a products stream 128 containing ethylene. The products stream 128 can contain 90 wt. % to 100 wt. % or at least any one of, equal to any one of, or between any two of 90 wt. %, 91 wt. %, 92 wt. %, 93 wt. %, 94 wt. %, 95 wt. %, 96 wt. %, 97 wt. %, 98 wt. %, 99 wt. % and 100 wt. % ethylene. The ethanol dehydration conditions can include a pressure 0 bar to 90 bar or at least any one of, equal to any one of, or between any two of 0 bar, 10 bar, 20 bar, 30 bar, 40 bar, 50 bar, 60 bar, 70 bar, 80 bar, and 90 bar, GHSV 1000 h⁻¹ to 3000 h⁻¹ or at least any one of, equal to any one of, or between any two of 1000 h⁻¹, 1500 h⁻¹, 2000 h⁻¹, 2500 h⁻¹ and 3000 h⁻¹ and/or a temperature 105° C. to 450° C. or at least any one of, equal to any one of, or between any two of 105° C., 150° C., 200° C., 250° C., 300° C., 350° C., 400° C., and 450° C. The ethanol dehydration catalysts can be an acid type catalyst. In some aspects, the acid type catalyst can be cesium doped silicotungstic acid supported on alumina. Non-limiting examples of ethanol dehydration catalysts that can be used in the context of the present invention include one or more of CeSiW₁₂O₄₀, RbSiW₁₂O₄₀, CePMo₁₂O₄₀, RbH₃PMo₁₂O₄₀, or a dehydration catalyst as described in Haider et al., Journal of Catalysis 286 (2012) 206-213.

In FIG. 1, the reactors, units and/or zones can include one or more heating and/or cooling devices (e.g., insulation, electrical heaters, jacketed heat exchangers in the wall) and/or controllers (e.g., computers, flow valves, automated values, inlets, outlets, etc.) that can be used to control the reaction temperature and pressure of the reaction mixture. While only one unit or zone is shown, it should be understood that multiple reactors or zones can be housed in one unit or a plurality of reactors housed in one heat transfer unit. In some aspects, the reactors can be a fixed bed reactor, moving bed reactors, trickle-bed reactor, rotating bed reactor, slurry reactors or fluidized bed reactor.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### EXAMPLES

As part of the disclosure of the present invention, specific examples are included below. The examples are for illustrative purposes only and are not intended to limit the invention. Those of ordinary skill in the art will readily recognize parameters that can be changed or modified to yield essentially the same results.

### Example 1

### Crystalline Cobalt Molybdenum Catalyst Preparation and Activity Evaluation

Catalyst preparation. Catalysts were prepared via co-precipitation method. Separate solutions of cobalt acetate (12.45 g, 100 ml d.H₂O) and ammonium heptamolybdate (8.45 g, 100 ml d.H₂O) were heated to 65° C. to dissolve the salts. While under stirring and the molybdenum solution heated at 65° C., the cobalt solution was added dropwise using a separating funnel and aged for 2 h. The solution was then filtered without washing and the dark purple precipitate was dried in an oven (110° C.) for 6 h. The catalyst precursor was calcined (500° C., static air, 10° C./min, 4 h) resulting in the cobalt molybdenum catalyst.

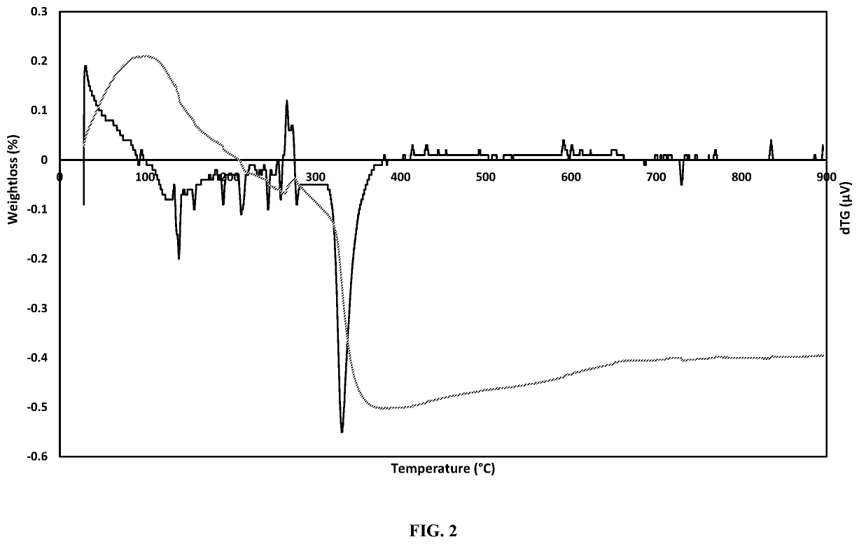

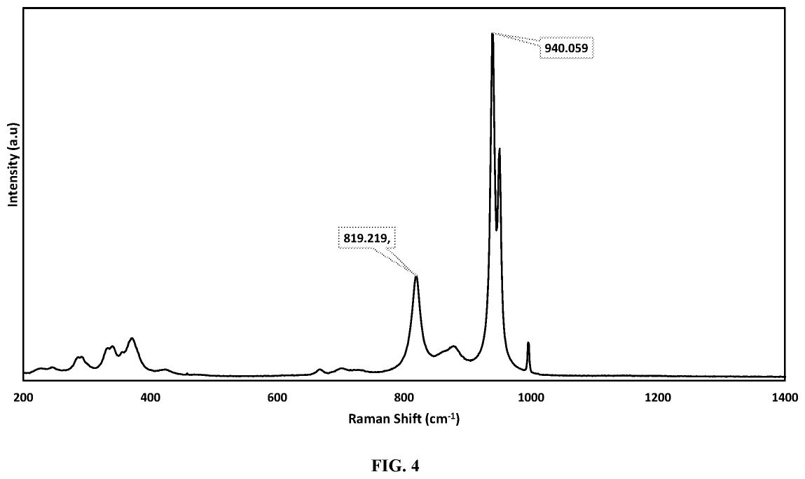

Catalyst Characterization. Catalyst characterization results are shown in FIG. 2-4. Thermogravimetric analysis (TGA) was used to assess the decomposition of the cobalt molybdate precursor to the final state catalyst (FIG. 2). TGA shows a weight loss from 300° C. to 400° C. showing the acetate evolving to CO2 shown by the negative peak (exothermic). This confirms the calcination at 500° C. would result in decomposition of the acetates and nitrates. X-ray powder diffraction (FIG. 3) shows reflections corresponding to monoclinic CoMoO₄ with α-CoMoP₄ and β-CoMoO₄ at a wt. % ratio 25:75. The peak at 11.7°, which is the main reflection of molybdenum trioxide is also observed suggesting phase separation as 1:1 Co:Mo ratio was used. The excess can also be seen in the Raman spectrum 819 cm⁻¹ (FIG. 4).

Catalyst activity and selectivity evaluation. The catalysts were evaluated for the activity and selectivity calculations along with short term as well as long studies of the catalyst stabilities. Prior to activity measurement, the catalysts were subjected to activation procedure, by reducing the catalyst with a H₂ (H₂, 100 ml/min, 350° C., 1° C./min, 16 h). Catalytic evaluation was carried out in high throughput fixed bed flow reactor setup housed in temperature-controlled system fitted with regulators to maintain pressure during the reaction. The products of the reactions were analyzed through online GC analysis. The evaluation was carried out under the following conditions unless otherwise mentioned elsewhere; 47.5% H₂/47.5% CO/5% N₂, 75 Bar, 300° C., 1° C./min, 48 h stabilization, 100 ml/min, 50% SiC mix.

The mass balance of the reactions is calculated to be 95±5%. Dehydration of alcohols produced can be carried out at a temperature above their boiling points and in the presence of and acid type catalyst for example cesium doped silicotungstic acid supported on alumina.

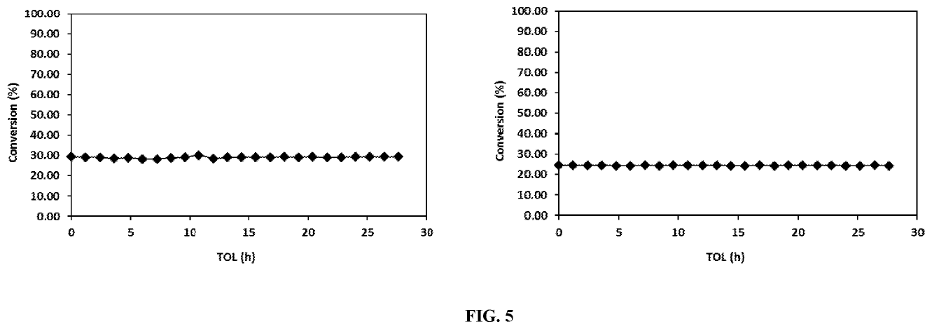

The results of catalyst testing are illustrated in FIG. 5 and Table 1. Duplicate sets of data show that the catalyst testing is reproducible. The catalyst is stable under reaction conditions, after a stabilization time of 48 h, with CO conversion at 24-30% (FIG. 5, Table 1). The catalyst produces substantial amount of MeOH and EtOH (ca. 60 mol. %) with small amount of propanol and butanol (ca. 10 mol. %) (Table 1).

TABLE 1
Product selectivity profile obtained from CO hydrogenation with the crystalline
cobalt molybdenum catalyst.

| | Conversion/Selectivity (mole %) | | | | | | | |
|---|---|---|---|---|---|---|---|---|
| TOS [h⁻¹] | Methanol | Ethanol | Propanol | Butanol | C₂-C₇ | CH₄ | CO₂ | Conversion |
| 0 | 25 | 25 | 8 | 7 | 19 | 0 | 15 | 30 |
| 2 | 33 | 28 | 7 | 6 | 10 | 0 | 16 | 30 |
| 4 | 25 | 25 | 9 | 8 | 19 | 0 | 14 | 30 |
| 5 | 32 | 30 | 8 | 7 | 10 | 0 | 13 | 30 |
| 6 | 30 | 34 | 8 | 7 | 8 | 0 | 13 | 30 |
| 7 | 30 | 28 | 9 | 9 | 10 | 0 | 14 | 30 |
| 8 | 29 | 29 | 9 | 8 | 10 | 0 | 15 | 30 |
| 10 | 31 | 30 | 9 | 9 | 9 | 0 | 12 | 30 |
| 11 | 28 | 31 | 6 | 6 | 18 | 0 | 11 | 30 |
| 12 | 33 | 26 | 8 | 7 | 11 | 0 | 15 | 30 |
| 13 | 30 | 30 | 7 | 7 | 10 | 0 | 16 | 30 |
| 14 | 30 | 29 | 8 | 7 | 15 | 0 | 11 | 30 |
| 16 | 29 | 35 | 7 | 7 | 10 | 0 | 12 | 30 |
| 17 | 34 | 30 | 6 | 5 | 10 | 0 | 15 | 30 |
| 18 | 28 | 27 | 6 | 6 | 20 | 0 | 13 | 30 |
| 19 | 30 | 29 | 10 | 9 | 10 | 0 | 12 | 30 |
| 20 | 28 | 35 | 7 | 7 | 8 | 0 | 15 | 30 |
| 22 | 30 | 27 | 4 | 4 | 20 | 0 | 15 | 30 |
| 23 | 28 | 27 | 8 | 7 | 13 | 0 | 17 | 30 |
| 24 | 30 | 29 | 8 | 8 | 10 | 0 | 15 | 30 |
| 25 | 29 | 33 | 3 | 3 | 18 | 0 | 14 | 30 |
| 26 | 33 | 30 | 5 | 4 | 12 | 0 | 16 | 30 |
| 28 | 30 | 29 | 7 | 7 | 10 | 0 | 17 | 30 |

In the context of the present invention, at least the following 20 embodiments are described. Embodiment 1 is a process for producing ethylene. The process includes: a) contacting a first stream containing methane with an oxidant and oxidizing at least a portion of the methane under conditions suitable to produce a second stream containing carbon monoxide (CO) and hydrogen (H₂); (b) contacting the second stream with a CO hydrogenation catalyst under conditions suitable to produce a third stream containing methanol and ethanol; (c) obtaining a fourth stream containing the ethanol, and a fifth stream containing methanol from the third stream; and (d) contacting the fourth stream with an ethanol dehydration catalyst under conditions suitable to dehydrate at least a portion of the ethanol and produce a products stream containing ethylene. Embodiment 2 is the process of embodiment 1, wherein the third stream further contains C2-C7 paraffins and carbon dioxide (CO₂) and the process further includes: (i) separating the third stream to obtain a first intermediate stream containing the methanol and ethanol and a second intermediate stream containing the C2-C7 paraffins and CO₂, and (ii) separating the first intermediate stream to obtain the fourth stream and the fifth stream. Embodiment 3 is the process of either of embodiments 1 or 2, wherein the CO hydrogenation catalyst contains a crystalline cobalt molybdenum catalyst. Embodiment 4 is the process of embodiment 3, wherein the crystalline cobalt molybdenum catalyst contains a monoclinic crystalline structure. Embodiment 5 is the process of embodiment 4, wherein the crystalline cobalt molybdenum catalyst is a monoclinic cobalt molybdenum oxide. Embodiment 6 is the process of embodiment 5, wherein the monoclinic cobalt molybdenum oxide is CoₓMo_{y}O_{z}, wherein x ranges from 0.5 to 1.5, y ranges from 0.5 to 1.5, and z ranges from 3.5 to 4.5. Embodiment 7 is the process of embodiment 6, wherein the monoclinic cobalt molybdenum oxide contains α-CoMoO4 and β-CoMoO₄ at a α-CoMoO₄ to β-CoMoO₄ wt. % ratio 15:85 to 35:65. Embodiment 8 is the process of any one of embodiments 1 to 7, wherein the CO hydrogenation catalyst is reduced and activated prior to contacting with the second stream. Embodiment 9 is the process of any one of embodiments 1 to 8, wherein the oxidant in step (a), is steam, oxygen (O₂), CO₂ or a combination thereof. Embodiment 10 is the process of any one of embodiments 1 to 9, wherein the oxidation of the at least a portion of the methane in the step (a) is catalyzed using a methane oxidation catalyst, wherein the methane oxidation catalyst contains one or more metals selected from La, Ni, Ru, Rh, Pd, Ir, and Pt, on a support containing alumina, silica, zirconia, ceria, titania, magnesium oxide, magnesium aluminate or any combination thereof. Embodiment 11 is the process of any one of embodiments 1 to 10, wherein the step (a) methane oxidation conditions include a pressure of 0 to 180 bar, GHSV of 5000 to 15000 and a temperature of 500 to 1600° C. Embodiment 12 is the process of any one of embodiments 1 to 11, wherein the molar ratio of the H₂ and CO in the second stream is 0.5:1 to 3:1. Embodiment 13 is the process of any one of embodiments 1 to 12, wherein the step (b) contacting conditions include a pressure of 25 to 90 bar, GHSV of 1000 to 3000 and a temperature of 150 to 450° C. Embodiment 14 is the process of any one of embodiments 2 to 13, wherein the third stream contains 20 mol. % to 40 mol. % methanol, 20 mol. % to 40 mol. % ethanol, 5 mol. % to 25 mol. % C2-C7 paraffins and 10 mol. % to 20 mol. % CO2. Embodiment 15 is the process of any one of embodiments 2 to 14, wherein in step (i) the third stream is separated by distillation using a distillation column and the first intermediate stream is obtained as a bottom distillate product and the second intermediate stream is obtain as a top distillate product. Embodiment 16 is the process of any one of embodiments 2 to 15, wherein in step (ii) first intermediate stream is separated by distillation using a distillation column and the fourth stream is obtain as a bottom distillate product and the fifth stream is obtained as a top distillate product. Embodiment 17 is the process of any one of embodiments 1 to 13, wherein the step (d) contacting conditions include a pressure of 0 to 90 bar, GHSV of 1000 to 3000 h⁻¹ and a temperature of 105 to 450° C. Embodiment 18 is the process of any one of embodiments 1 to 17, wherein the dehydration catalyst in step (d) is an acid type catalyst. Embodiment 19 is the process of embodiment 18, wherein the acid type catalyst is cesium doped silicotungstic acid supported on alumina. Embodiment 20 is the process of any one of embodiments 1 to 19, wherein the methane in the first stream is obtained from a refinery, petroleum by product, renewable feedstock, or a combination thereof

1. A process for producing ethylene, the process comprising:
(a) contacting a first stream comprising methane with an oxidant and oxidizing at least a portion of the methane under conditions suitable to produce a second stream comprising carbon monoxide (CO) and hydrogen (H₂);
(b) contacting the second stream with a CO hydrogenation catalyst under conditions suitable to produce a third stream comprising methanol and ethanol;
(c) obtaining a fourth stream comprising the ethanol, and a fifth stream comprising methanol from the third stream; and
(d) contacting the fourth stream with an ethanol dehydration catalyst under conditions suitable to dehydrate at least a portion of the ethanol and produce a products stream comprising ethylene.

2. The process of claim 1, wherein the third stream further comprises C2-C7 paraffins and carbon dioxide (CO₂) and the process further comprises:
(i) separating the third stream to obtain a first intermediate stream containing the methanol and ethanol and a second intermediate stream containing the C2-C7 paraffins and CO₂; and
(ii) separating the first intermediate stream to obtain the fourth stream and the fifth stream.

3. The process of claim 1, wherein the CO hydrogenation catalyst comprises a crystalline cobalt molybdenum catalyst.

4. The process of claim 3, wherein the crystalline cobalt molybdenum catalyst comprises a monoclinic crystalline structure.

5. The process of claim 4, wherein the crystalline cobalt molybdenum catalyst is a monoclinic cobalt molybdenum oxide.

6. The process of claim 5, wherein the monoclinic cobalt molybdenum oxide is CoₓMo_{y}O_{z},
wherein x ranges from 0.5 to 1.5, y ranges from 0.5 to 1.5, and z ranges from 3.5 to 4.5.

7. The process of claim 6, wherein the monoclinic cobalt molybdenum oxide comprises α-CoMoO4 and β-CoMoO₄ at a α-CoMO₄ to β-CoMO₄ wt. % ratio 15:85 to 35:65.

8. The process of claim 1, wherein the CO hydrogenation catalyst is reduced and activated prior to contacting with the second stream.

9. The process of claim 1, wherein the oxidant in step (a), is steam, oxygen (O₂), CO₂ or a combination thereof.

10. The process of claim 1, wherein the oxidation of the at least a portion of the methane in the step (a) is catalyzed using a methane oxidation catalyst, wherein the methane oxidation catalyst comprises one or more metals selected from La, Ni, Ru, Rh, Pd, Ir, and Pt, on a support comprising alumina, silica, zirconia, ceria, titania, magnesium oxide, magnesium aluminate or any combination thereof.

11. The process of claim 1, wherein the step (a) methane oxidation conditions comprise a pressure of 0 to 180 bar, GHSV of 5000 to 15000 h⁻¹ and a temperature of 500 to 1600° C.

12. The process of claim 1, wherein the molar ratio of the H₂ and CO in the second stream is 0.5:1 to 3:1.

13. The process of claim 1, wherein the step (b) contacting conditions comprise a pressure of 25 to 90 bar, GHSV of 1000 to 3000 h⁻¹, and a temperature of 150 to 450° C.

14. The process of claim 2, wherein the third stream comprises 20 mol. % to 40 mol. % methanol, 20 mol. % to 40 mol. % ethanol, 5 mol. % to 25 mol. % C2-C7 paraffins and 10 mol. % to 20 mol. % CO₂.

15. The process of claim 2, wherein in step (i) the third stream is separated by distillation using a distillation column and the first intermediate stream is obtained as a bottom distillate product and the second intermediate stream is obtain as a top distillate product.

16. The process of claim 2, wherein in step (ii) first intermediate stream is separated by distillation using a distillation column and the fourth stream is obtain as a bottom distillate product and the fifth stream is obtained as a top distillate product.

17. The process of claim 1, wherein the step (d) contacting conditions comprise a pressure of 0 to 90 bar, GHSV of 1000 to 3000 h⁻¹ and a temperature of 105 to 450° C.

18. The process of claim 1, wherein the dehydration catalyst in step (d) is an acid type catalyst.

19. The process of claim 18, wherein the acid type catalyst is cesium doped silicotungstic acid supported on alumina.

20. The process of claim 1, wherein the methane in the first stream is obtained from a refinery, petroleum by product, renewable feedstock, or a combination thereof.
